(19) United States
(12) Patent Application Publication
(10) Pub. No.: US 2014/0187833 A1
(43) Pub. Date: Jul. 3, 2014

(54) INTEGRATED PROCESS FOR THE PREPARATION OF OLEFINS

(71) Applicant: Shell Oil Company, Houston, TX (US)

(72) Inventors: Leslie Andrew CHEWTER, Amsterdam (NL); Sivakumar SADASIVAN VIJAYAKUMARI, Amsterdam (NL); Jeroen VAN WESTRENEN, Amsterdam (NL)

(73) Assignee: Shell Oil Company, Houston, TX (US)

(21) Appl. No.: 14/135,952

(22) Filed: Dec. 20, 2013

(30) Foreign Application Priority Data
Dec. 31, 2012 (EP) 12199825.6

(51) Int. Cl. C07C 7/13; C07C 4/04

(52) U.S. Cl. CPC C07C 7/13; C07C 4/04
USPC 585/304

(57) ABSTRACT

The invention provides a process for preparing olefins, comprising: (a) reacting an oxygenate and/or olefinic feed in a first reactor in the presence of a molecular sieve catalyst to form a first effluent comprising olefins; (b) fractionating at least part of the first effluent into an olefinic product fraction comprising ethylene and propylene and an olefinic product fraction comprising olefins containing 4 or more carbon atoms; (c) subjecting a paraffin-containing hydrocarbon feedstock in a second reactor to a steam cracking process to form a second effluent comprising olefins including butadiene; (d) fractionating the second effluent into an olefinic product fraction comprising ethylene and/or propylene and an olefinic product fraction comprising mono-olefins containing 4 or more carbon atoms; and (e) recycling the olefinic product fraction comprising at least part of the ethylene and/or propylene as obtained in step (d) to the reactor in step (a).

This application claims the benefit of European Application No. 12199825.6 filed Dec. 31, 2012, which is incorporated herein by reference.

### FIELD OF THE INVENTION

The present invention relates to an integrated process for the preparation of olefins.

### BACKGROUND OF THE INVENTION

There is an increasing demand for olefins in the chemical industry. In particular olefins such as ethylene, propylene, and butadiene are of commercial importance since they are used as building block chemicals for the preparation of petrochemicals and polymers. Ethylene and propylene are used for the production of polyethylene and polypropylene respectively, whereas butadiene is used for the production of polybutadiene and styrene-butadiene-rubber (SBR).

Processes for the preparation of olefins are known in the art. A major process for the preparation of olefins is the steam cracking of a paraffin-containing hydrocarbon feedstock such as naphtha, gas oil, ethane, propane and butane. Other suitable and more recently developed processes for the preparation of olefins include oxygenate-to-olefins (OTO) processes and olefin cracking processes (OCP).

In US 2010/0206771 A1, an integrated process for producing hydrocarbons, in particular C₂-C₄ has been described, using a combined plant with a steam cracker and a reactor for converting an educt mixture which includes steam and at least one oxygenate, wherein the respective intermediate product streams of the steam cracker and the reactor are combined completely.

Such a process leaves room for improvement in terms of flexibility in the amounts and ratios of ethylene and/or propylene to be produced especially on flexibility to cope with market demand fluctuations.

An object of the present invention is to provide an integrated process of a steam cracker and an OTO or OCP reactor for producing olefins such as ethylene and/or propylene in more flexible amounts and ratios in line with olefin market demand for polyethylene and polypropylene requiring different propylene to ethylene monomer ratios per grade.

### SUMMARY OF THE INVENTION

It has now been found that this can be established when the various product streams are integrated in a particular manner.

Accordingly, the present invention relates to an integrated process for the preparation of olefins, which process comprises the steps of:
(a) reacting an oxygenate and/or olefinic feed in a first reactor in the presence of a molecular sieve catalyst to form a first effluent which comprises olefins;
(b) fractionating at least part of the first effluent into an olefinic product fraction comprising at least ethylene and propylene and an olefinic product fraction which comprises olefins containing 4 or more carbon atoms;
(c) subjecting a paraffin-containing hydrocarbon feedstock in a second reactor to a steam cracking process to form a second effluent which comprises olefins including butadiene;
(d) fractionating at least part of the second effluent into an olefinic product fraction comprising at least ethylene and/or propylene and an olefinic product fraction comprising mono-olefins containing 4 or more carbon atoms; and
(e) recycling at least part of the olefinic product fraction comprising at least part of the ethylene and/or propylene as obtained in step (d) to the reactor in step (a).

The process according to the present invention allows highly attractively and depending on market demand adjusting the production of alternatingly homo-polymer polypropylene and co-polymer propylene in which ethylene is used as a co-polymer. In addition, in case of a shut-down of a polypropylene unit, propylene can attractively be recycled and C4 and C5 olefins can be recovered as intermediate products.

### BRIEF DESCRIPTION OF THE DRAWING

FIG. 1 shows schematically an embodiment of the present invention.

### DETAILED DESCRIPTION OF THE INVENTION

In step (a) of the present process an oxygenate and/or olefinic feed is reacted in a first reactor in the presence of a molecular sieve catalyst to form a first effluent which comprises olefins.

In step (a), preferably oxygenates are converted into olefins.

The reactor to be used in step (a) can be an OTO reaction zone wherein the oxygenate feed is contacted with an oxygenate conversion catalyst under oxygenate conversion conditions, to obtain the first effluent comprising lower olefins. Reference herein to an oxygenate feed is to an oxygenate-comprising feed. In the OTO reaction zone, at least part of the feed is converted into a product containing one or more olefins, preferably including lower olefins, in particular ethylene and typically propylene.

The oxygenate used in the process according to the invention is preferably an oxygenate which comprises at least one oxygen-bonded alkyl group. The alkyl group preferably is a C₁-C₅ alkyl group, more preferably C₁-C₄ alkyl group, i.e. comprises 1 to 5, respectively, 4 carbon atoms; more preferably the alkyl group comprises 1 or 2 carbon atoms and most preferably one carbon atom. Examples of oxygenates that can be used in the oxygenate feed include alcohols and ethers. Examples of preferred oxygenates include alcohols, such as methanol, ethanol, propanol; and dialkyl ethers, such as dimethylether, diethylether, methylethylether. Preferably, the oxygenate is methanol or dimethylether, or a mixture thereof. More preferably, the oxygenate comprises methanol or dimethylether.

Preferably the oxygenate feed comprises at least 50 wt % of oxygenate, in particular methanol and/or dimethylether, based on total hydrocarbons, more preferably at least 70 wt %.

The oxygenate feed can comprise an amount of diluent, such as nitrogen and water, preferably in the form of steam. In one embodiment, the molar ratio of oxygenate to diluent is between 10:1 and 1:10, preferably between 4:1 and 1:2, in particular when the oxygenate is methanol and the diluent is water (steam).

A variety of OTO processes is known for converting oxygenates such as for instance methanol or dimethylether to an olefin-containing product, as already referred to above. One such process is described in WO 2006/020083.

The molecular sieve catalyst to be used in step (a) suitably comprises one or more zeolite catalysts and/or one or more SAPO catalysts. Molecular sieve catalysts typically also include binder materials, matrix material and optionally fillers. Suitable matrix materials include clays, such as kaolin. Suitable binder materials include silica, alumina, silica-alumina, titania and zirconia, wherein silica is preferred due to its low acidity.

The molecular sieve catalyst preferably has a molecular framework of one, preferably two or more corner-sharing [TiO₄] tetrahedral units, more preferably, two or more [SiO₄] and/or [AlO₄] tetrahedral units. These silicon and/or aluminum based molecular sieves and metal containing silicon and/or aluminum based molecular sieves have been described in detail in numerous publications including for example, U.S. Pat. No. 4,567,029. In a preferred embodiment, the molecular sieve catalysts have 8-, 10- or 12-ring structures and an average pore size in the range of from about 3 Å to 15 Å.

Preferably, the amount of zeolite is suitably from 20 to 50 wt %, preferably from 35 to 45 wt %, based on total weight of the molecular sieve catalyst composition.

Suitable zeolites include those of the ZSM group, in particular of the MFI type, such as ZSM-5, the MTT type, such as ZSM-23, the TON type, such as ZSM-22, the MEL type, such as ZSM-11, the FER type. Other suitable zeolites are for example zeolites of the STF-type, such as SSZ-35, the SFF type, such as SSZ-44 and the EU-2 type, such as ZSM-48.

Preferred zeolites comprise more-dimensional zeolites, in particular of the MFI type, more in particular ZSM-5, or of the MEL type, such as zeolite ZSM-11. The zeolite having more-dimensional channels has intersecting channels in at least two directions. So, for example, the channel structure is formed of substantially parallel channels in a first direction, and substantially parallel channels in a second direction, wherein channels in the first and second directions intersect. Intersections with a further channel type are also possible. Preferably, the channels in at least one of the directions are 10-membered ring channels. A preferred MFI-type zeolite has a Silica-to-Alumina ratio (SAR) of at least 60, preferably at least 80.

Particular preferred catalysts include catalysts comprising one or more zeolite having one-dimensional 10-membered ring channels, i.e. one-dimensional 10-membered ring channels, which are not intersected by other channels.

In a particular embodiment of the present invention use is made of a molecular sieve catalyst which comprises ZSM-5 and/or ZSM-11.

Preferred examples are zeolites of the MTT and/or TON type.

Preferably, the catalyst comprises at least 40 wt %, preferably at least 50% wt of such zeolites based on total zeolites in the catalyst.

In a particularly preferred embodiment the molecular sieve catalyst comprises in addition to one or more one-dimensional zeolites having 10-membered ring channels, such as of the MTT and/or TON type, a more-dimensional zeolite, in particular of the MFI type, more in particular ZSM-5, or of the MEL type, such as zeolite ZSM-11.

The molecular sieve catalyst can comprise at least 1 wt %, based on total molecular sieve in the oxygenate conversion catalyst, of the second molecular sieve having more-dimensional channels, preferably at least 5 wt %, more preferably at least 8 wt %.

The present molecular sieve catalyst may comprise phosphorus as such or in a compound, i.e. phosphorous other than any phosphorus included in the framework of the molecular sieve. It is preferred that an MEL or MFI-type zeolite comprising catalyst additionally comprises phosphorus. The phosphorus may be introduced by pre-treating the MEL or MFI-type zeolites prior to formulating the catalyst and/or by post-treating the formulated catalyst comprising the MEL or MFI-type zeolites. Preferably, the present molecular sieve catalyst comprising MEL or MFI-type zeolites comprises phosphorus as such or in a compound in an elemental amount of from 0.05-10 wt % based on the weight of the formulated catalyst. A particularly preferred catalyst comprises phosphorus-treated MEL or MFI-type zeolites having SAR of in the range of from 60 to 150, more preferably of from 80 to 100. An even more particularly preferred catalyst comprises phosphorus-treated ZSM-5 having SAR of in the range of from 60 to 150, more preferably of from 80 to 100.

It is preferred that the molecular sieves in the hydrogen form are, e.g., HZSM-22, HZSM-23, and HZSM-48, HZSM-5. Preferably at least 50% w/w, more preferably at least 90% w/w, still more preferably at least 95% w/w and most preferably 100% of the total amount of molecular sieve catalyst used is in the hydrogen form. It is well known in the art how to produce such molecular sieve catalysts in the hydrogen form.

The molecular sieve catalyst particles can have any shape known to the skilled person to be suitable for this purpose, for it can be present in the form of spray dried catalyst particles, spheres, tablets, rings, extrudates, etc. Extruded catalysts can be applied in various shapes, such as, cylinders and trilobes. Spherical particles are normally obtained by spray drying. Preferably the average particle size is in the range of 1-500 μm, preferably 50-100 μm.

Preferably, the zeolite comprises a zeolite having 10-membered ring channels. Preferred zeolites include zeolites of the MTT-type, the TON-type, the MFI-type or the MEL-type.

The reaction conditions of the oxygenate conversion in step (a) include a reaction temperature of 350 to 1000° C., suitably from 350 to 750° C., preferably from 450 to 750° C., more preferably from 450 to 700° C., even more preferably 500 to 650° C.; and a pressure suitably from 1 bara to 50 bara, preferably from 1-15 bara, more preferably from 1-4 bara, even more preferably from 1.1-3 bara, and most preferably in from 1.3-2 bara.

Suitably, the oxygenate-comprising feed is preheated to a temperature in the range of from 120 to 550° C., preferably 250 to 500° C. prior to introducing it into the reactor in step (a).

Preferably, in addition to the oxygenate, an olefinic co-feed is provided along with and/or as part of the oxygenate feed. Reference herein to an olefinic co-feed is to an olefin-comprising co-feed. The olefinic co-feed preferably comprises C4 and higher olefins, more preferably C4 and C5 olefins. Preferably, the olefinic co-feed comprises at least 25 wt %, more preferably at least 50 wt %, of C4 olefins, and at least a total of 70 wt % of C4 hydrocarbon species. The olefinic co-feed can also comprise propylene.

The reaction in step (a) may suitably be operated in a fluidized bed or moving bed, e.g. a dense, turbulent or fast fluidized bed or a riser reactor or downward reactor system, and also in a fixed bed reactor or a multitubular fixed bed reactor. A fluidized bed or moving bed, e.g. a turbulent fluidized bed, fast fluidized bed or a riser reactor system are preferred. In addition, hybrid reactors such as part turbulent, part fast and/or part riser reactors can be used.

The superficial velocity of the gas components in a dense fluidized bed will generally be from 0 to 1 m/s; the superficial velocity of the gas components in a turbulent fluidized bed will generally be from 1 to 3 m/s; the superficial velocity of the gas components in a fast fluidized bed will generally be from 3 to 5 m/s; and the superficial velocity of the gas components in a riser reactor will generally be from 5 to about 25 m/s.

It will be understood that dense, turbulent and fast fluidized beds will include a dense lower reaction zone with densities generally above 300 kg/m³. Moreover, when working with a fluidized bed several possible configurations can be used: (a) co-current flow meaning that the gas (going upward) and the catalyst travels through the bed in the same direction, and (b) countercurrent, meaning that the catalyst is fed at the top of the bed and travels through the bed in opposite direction with respect to the gas, whereby the catalyst leaves the vessel at the bottom. In a conventional riser reactor system the catalyst and the vapors will travel co-currently.

Suitably, a fluidized bed, in particular a turbulent fluidized bed system is used. Suitably, in such a moving bed reactor the oxygenate feed is contacted with the molecular sieve catalyst at a weight hourly space velocity of at least 1 hr⁻¹, suitably from 1 to 1000 hr⁻¹, preferably from 1 to 500 hr⁻¹, more preferably 1 to 250 hr⁻¹, even more preferably from 1 to 100 hr⁻¹, and most preferably from 1 to 50 hr⁻¹.

The reactor in step (a) can also be an OCP reaction zone wherein an olefinic feed is contacted with an olefin conversion catalyst under olefin conversion conditions, to form the first effluent comprising lower olefins.

Suitably, the olefinic feed comprises C4+ olefins that will be converted to ethylene and/or propylene by contacting such a feed with the zeolite-comprising catalyst. Preferably, the olefinic feed is contacted with the zeolite-comprising catalyst in step (a) at a reaction temperature of 350 to 1000° C., preferably from 375 to 750° C., more preferably 450 to 700° C., even more preferably 500 to 650° C.; and a pressure from 1 bara to 50 bara, preferably from 1-15 bara. Optionally, such olefinic feed also contains a diluent. Examples of suitable diluents include, but are not limited to, diluents such as water or steam, nitrogen, paraffins and methane. Under these conditions, at least part of the olefins in the olefinic feed are converted to further ethylene and/or propylene.

Particular preferred molecular sieve catalysts for the OCP reaction are catalysts comprising at least one zeolite selected from MFI, MEL, TON and MTT type zeolites, more preferably at least one of ZSM-5, ZSM-11, ZSM-22 and ZSM-23 zeolites.

Also an OCP process may suitably be operated in a fluidized bed or moving bed, e.g. a fast fluidized bed or a riser reactor or a downward reactor system, and also in a fixed bed reactor or a tubular reactor. A fluidized bed or moving bed, e.g. a fast fluidized bed or a riser reactor system are preferred.

Suitably, the first effluent as obtained in step (a) comprises less than 1.0 wt % butadiene, preferably less than 0.50 wt %, based on the total weight of hydrocarbons containing 4 carbon atoms present in the first effluent.

In step (b) at least part of the first effluent is fractionated into an olefinic product fraction comprising at least ethylene and propylene and an olefinic product fraction which comprises olefins containing 4 or more carbon atoms.

Suitably, at least part of the olefinic product fraction which comprises olefins containing 4 or more carbon atoms is recycled to step (a). If desired, at least part of the olefinic product fraction which comprises olefins containing 4 or more carbon atoms can be withdrawn as olefinic product. Preferably, the entire olefinic product fraction which comprises olefins containing 4 or more carbon atoms is recycled to step (a).

The skilled person will understand that step (b) can be carried out using separation units in a variety of separation methods including, but not limited to distillation, extractive distillation, membranes, liquid-liquid extraction, absorption etc. Suitable separation units include distillation columns such as demethanisers, deethanisers, depropanisers, debutanisers, propylene/propane splitters and ethylene/ethane splitters. These separation units can be used in various sequences, allowing at least part of the olefinic product fraction which comprises olefins containing 4 or more carbon atoms to be recycled to step (a). Preferably, step (b) is carried out by passing the first effluent to a debutaniser unit and retrieving the olefinic product fraction comprising at least ethylene and propylene as the top fraction and the olefinic product fraction which comprises olefins containing 4 or more carbon atoms as the bottom fraction.

If desired, the olefinic product fraction which comprises olefins containing 4 or more carbon atoms as obtained in step (b) can be fractionated to obtain at least an olefinic product fraction comprising C4 olefins and an olefinic product fraction comprising olefins having 5 or more carbon atoms. Suitably, at least part of the olefinic product fraction comprising C₄ olefins so obtained is recycled to step (a). Preferably, the entire olefinic product fraction comprising c4 olefins so obtained is recycled to step (a).

Suitably, at least part of the olefinic product fraction comprising olefins having 5 or more carbon atoms so obtained can be subjected to an olefin cracking process to form lighter olefins. Suitably, the entire olefinic product fraction comprising olefins having 5 or more carbon atoms so obtained can be subjected to an olefin cracking process to form lighter olefins. Preferably, the C₄, C₅ and C₆ fractions are subjected to the olefin cracking process in step (a)

In step (c), a paraffin-containing hydrocarbon feedstock is subjected in a second reactor to a steam cracking process to form a second effluent which comprises olefins including butadiene.

Steam cracking processes that can be applied in step (c) include those known in the art. Suitable steam cracking processes to be used in step (c) are, for example, described in U.S. Pat. No. 3,365,387 and U.S. Pat. No. 4,061,562. In a steam cracking process the hydrocarbons are pyrolyzed in the presence of steam to form light olefins such as ethylene and propylene, and heavier products such as gas oil and gasoline.

In the steam cracking process in step (c), the paraffin-containing hydrocarbon feedstock is contacted with steam at a temperature and pressure to form the second effluent which comprises olefins such as ethylene and propylene, including butadiene.

Suitably, the steam cracking process in step (c) is conducted at a temperature from 750 to 920° C., preferably from 760 to 900° C., more preferably from 770 to 890° C., even more preferably 780 to 880° C.; and a pressure of from 1 to 5 bara, preferably from 1.1 to 2.5 bara, more preferably from 1.5 to 2.5 bara.

The paraffin-containing hydrocarbon feedstock to be steam cracked can be chosen from a variety of petroleum fractions. Preferably, the paraffin-containing hydrocarbon feedstock comprises at least one of ethane, propane, butane and/or at least one of a naphtha, kerosene, gasoil or hydrowax.

In step (d), at least part of the second effluent as obtained in step (c) is fractionated into an olefinic product fraction comprising at least ethylene and propylene and an olefinic fraction comprising olefins containing 4 or more carbon atoms.

Suitably, in step (d) at least part of the second effluent as obtained in step (c) is fractionated into an olefinic product fraction comprising at least ethylene, an olefinic product fraction comprising at least propylene and an olefinic product fraction comprising olefins containing 4 or more carbon atoms; and at least part of the olefinic product fraction comprising at least propylene is recycled to the reactor in step (a).

In another embodiment, in step (d) at least part of the second effluent is fractionated into an olefinic product fraction comprising at least ethylene, an olefinic product fraction comprising at least propylene and an olefinic product fraction comprising olefins containing 4 or more carbon atoms; and at least part of the olefinic product fraction comprising at least ethylene is recycled to the reactor in step (a).

The skilled person will understand that step (d) can be carried out using separation units in a variety of separation methods including, but not limited to distillation, extractive distillation, membranes, liquid-liquid extraction, absorption etc. Suitable separation units include distillation columns such as demethanisers, deethanisers, depropanisers, debutanisers, propylene/propane splitters and ethylene/ethane splitters. These separation units can be used in various sequences allowing at least part of the olefinic product fraction comprising at least ethylene and/or propylene as obtained in step (d) to be recycled to step (a).

Suitably, the fractionating in steps (b) and (d) are carried out in a shared fractionating system. This is especially attractive if butadiene is either hydrogenated or if the combined stream is subjected to butadiene extraction before the C4 mono-olefins and saturates are recycled to step (a).

Suitably, the product fraction that includes butadiene as obtained in step (c) contains in the range of from 30 to 60 wt % butadiene, based on the total weight of hydrocarbons containing 4 carbon atoms present in the product fraction that comprises butadiene.

The second effluent as obtained in step (c) contains a variety of contaminants and C₄+ components in addition to ethylene and propylene, which contaminants and C₄+ components need to be separated from ethylene and propylene to obtain chemical or polymerization grade ethylene and propylene. The second effluent as obtained in step (c) can, for instance, suitably be cooled using a primary fractionation unit followed by a quench unit to form a cooled product stream, which can suitably be passed to one or more fractionation units after compression and removal of CO₂ in a caustic wash and water through passing dryer beds. Suitably, the cooled product steam thus obtained is separated into one or more of the following fractions: a C₅+ fraction, a C₄ fraction, a C₃ fraction, a C₂ fraction, and a light fraction comprising lighter contaminants such as methane, light hydrocarbons and/or inerts. Preferably, the C₄ fraction which comprises a mixture of C₄ hydrocarbons is subsequently subjected to a butadiene extraction treatment to form a butadiene-enriched C₄ product stream and a butadiene-depleted C₄ product stream.

Preferably, at least part of the butadiene-depleted product stream so obtained is recycled to step (a). More preferably, the entire butadiene-depleted product stream so obtained is recycled to step (a).

The butadiene extraction treatment can suitably be any butadiene extraction treatment known in the art. For instance, the butadiene extraction treatment can be carried out by extractive distillation using acetonitrile as the solvent.

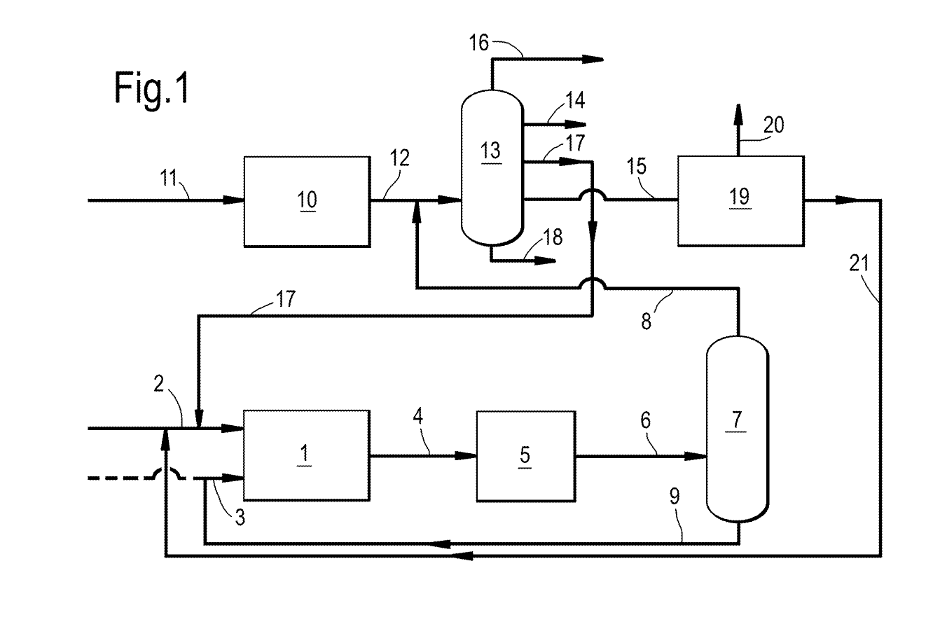

In FIG. 1, an oxygenate and/or olefinic feedstock is fed to a reactor 1 via line 2. Optionally, an olefinic co-feed is fed to the reactor as well, via line 3. In the reactor system 1, the oxygenate and/or olefinic feedstock and the olefinic co-feed are allowed to react in the presence of an oxygenate and/or olefin conversion catalyst to form a first effluent which comprises olefins. The olefins and portions of the at least partially coked catalyst are passed via line 4 to a solids-gas separator, such as a cyclone 5 in which the olefins containing effluent is separated from the at least partially coked catalyst. The olefins are recovered and passed via line 6 to a fractionation system 7 to form an olefinic product fraction comprising at least ethylene and propylene and an olefinic product fraction which comprises olefins containing 4 or more carbon atoms. The olefinic product fraction comprising at least ethylene and propylene is recovered in line 8 and the olefinic product fraction which comprises olefins containing 4 or more carbon atoms is recovered in line 9. At least part of the olefinic product which comprises olefins containing 4 or more carbon atoms stream is recycled to the reactor 1. A paraffin-containing hydrocarbon feedstock is fed to a reactor 10 via line 11. In reactor 10 the gas oil is steam cracked to form a second effluent which comprises olefins including butadiene. The second effluent is recovered and passed via line 12 to a fractionation section 13. In this embodiment the fractionation section is shown to produce an ethylene-rich product fraction in line 14, a C₄ fraction in line 15, a light fraction in overhead line 16 comprising lighter contaminants such as methane, light hydrocarbons and/or inerts, a propylene-rich product fraction in line 17 and a C₅+ hydrocarbon rich stream in line 18. CO₂ can be removed from such streams by way of caustic washing and drying the product streams so obtained. Likewise heavy hydrocarbons are condensed and removed through the primary fractionator. However, all such streams have not been depicted in FIG. 1. The propylene-rich product fraction is recycled to reactor 1. A bleed stream for C₄ olefins can be applied in case of recovery as intermediate products and reduced propylene product off take, but is not shown in FIG. 1. It is observed that homo-polymer polypropylene has another market demand than high impact polypropylene for which ethylene is used as a co-polymer. The present process enables very attractively the production of homo-polymer polypropylene and high impact polypropylene for which ethylene is used as a co-polymer in market adjustable amounts and, optionally in an alternating fashion in the same polymer propylene unit. The C₄ fraction so obtained is passed via line 15 to a butadiene extraction unit 20 to form a butadiene-enriched product stream which is recovered via line 21 and a butadiene-depleted product stream which is recycled to reactor 1 via line 22.

What is claimed is:

1. An integrated process for the preparation of olefins, which process comprises the steps of:
(a) reacting an oxygenate and/or olefinic feed in a first reactor in the presence of a molecular sieve catalyst to form a first effluent which comprises olefins;
(b) fractionating at least part of the first effluent into an olefinic product fraction comprising at least ethylene and propylene and an olefinic product fraction which comprises olefins containing 4 or more carbon atoms;
(c) subjecting a paraffin-containing hydrocarbon feedstock in a second reactor to a steam cracking process to form a second effluent which comprises olefins including butadiene;
(d) fractionating at least part of the second effluent into an olefinic product fraction comprising at least ethylene and/or propylene and an olefinic product fraction comprising mono-olefins containing 4 or more carbon atoms; and
(e) recycling at least part of the olefinic product fraction comprising at least part of the ethylene and/or propylene as obtained in step (d) to the reactor in step (a).

2. The process of claim 1, wherein in step (d) at least part of the second effluent is fractionated into an olefinic product fraction comprising at least ethylene, an olefinic product fraction comprising at least propylene and an olefinic product fraction comprising olefins containing 4 or more carbon atoms; and at least part of the olefinic product fraction comprising at least propylene is recycled to the reactor in step (a).

3. The process of claim 1, wherein in step (d) at least part of the second effluent is fractionated into an olefinic product fraction comprising at least ethylene, an olefinic product fraction comprising at least propylene and an olefinic product fraction comprising olefins containing 4 or more carbon atoms; and at least part of the olefinic product fraction comprising at least ethylene is recycled to the reactor in step (a).

4. The process of claim 1, wherein step (b) is carried out by passing the first effluent to a fractionating unit and retrieving the olefinic product fraction comprising at least ethylene and propylene as the top fraction and the olefinic product fraction which comprises olefins containing 4 or more carbon atoms as the bottom fraction.

5. The process of claim 1, wherein at least part of the olefinic product fraction which comprises olefins containing 4 or more carbon atoms is recycled to the reactor in step (a).

6. The process of claim 2, wherein the entire olefinic product fraction which comprises at least propylene or at least ethylene is recycled to the reactor in step (a).

7. The process of claim 2, wherein the product fraction which comprises butadiene as obtained in step (c) is subjected to a butadiene extraction treatment to form a butadiene-enriched product stream and butadiene-depleted product stream.

8. The process of claim 7, wherein at least part of the butadiene-depleted product stream is recycled to the reactor in step (a).

9. The process of claim 2, wherein the product fraction that includes butadiene as obtained in step (c) contains in the range of from 30 to 60 wt % butadiene, based on the total weight of hydrocarbons containing 4 carbon atoms present in the product fraction that includes butadiene.

10. The process of claim 1, wherein the first effluent as obtained in step (a) comprises less than 1.0 wt % butadiene based on the total weight of hydrocarbons containing 4 carbon atoms present in the first effluent.

11. The process of claim 1, wherein the molecular sieve catalyst in step (a) comprises an MFI or MEL zeolite.

12. The process of claim 1, wherein the oxygenate feed in step (a) comprises methanol and/or dimethylether.

13. The process of claim 1, wherein the paraffin-containing hydrocarbon feedstock in step (c) comprises at least one of ethane, propane, butane and/or at least one of naphtha, kerosene, gasoil or hydrowax.

14. The process of claim 1, wherein the reaction in step (a) is conducted at a temperature from 350 to 750° C., and a pressure of from 1 to 15 bara.

15. The process of claim 1, wherein the steam cracking process in step (c) is conducted at a temperature from 750 to 920° C. and a pressure of from 1 to 5 bara.
